(19) United States
(12) Patent Application Publication
(10) Pub. No.: US 2012/0059347 A1
(43) Pub. Date: Mar. 8, 2012

(54) PLUNGER CONTROL DEVICE FOR A SYRINGE AND ASSOCIATED METHOD

(76) Inventors: David Arthur Freed, New York, NY (US); Bochong Li, Durham, NC (US); Jeremy Eron Fetvedt, Raleigh, NC (US); Pia Felicitas Hoellerbauer, Delaware, OH (US); Michael Richard, Durham, NC (US); Nicholas Adam Ramey, Durham, NC (US)

(21) Appl. No.: 12/875,492

(22) Filed: Sep. 3, 2010

(51) Int. Cl. A61M 5/315

(52) U.S. Cl. 604/500; 604/218; 604/228

(57) ABSTRACT

An apparatus and associated method are provided for controlling the plunging action of a syringe. A plunger control device includes an attachment member adapted to fixedly engage a syringe barrel. A translating member is slideably engaged with the attachment member. The translating member includes an elongate guide member slideably engaged with the attachment member such that the translating member is slideable with respect thereto between a proximal end and a distal end of the elongate guide member. The translating member further includes a plunger attachment portion disposed about the distal end of the elongate guide member and adapted to receive a portion of a syringe plunger for coupling thereto. The translating member further includes an engagement member extending from the proximal end of the elongate guide member so as to be spaced-apart from the syringe barrel when the attachment member is engaged with the syringe barrel.

### BACKGROUND OF THE DISCLOSURE

1. Field of the Disclosure

The present disclosure relates to medical devices and, more particularly, to a device capable of being coupled to a syringe or components thereof for controlling the plunging action of the syringe, and an associated method.

2. Description of Related Art

A conventional syringe structure includes a needle held at its base by a hub that is mounted at the leading end of a barrel. A plunger is slideably mounted in the barrel, and a piston having annular seals is provided at the leading end of the plunger. In a syringe used for injection, displacement of the piston/plunger in a trailing-to-leading direction drives a liquid fluid out of the barrel and through the needle into the patient's tissue or other bodily portion. In this regard, the plunger is retracted to accomplish aspiration, and thereafter advanced to accomplish injection. When a conventional syringe is used in this manner, the physician must develop a technique whereby the needle remains properly positioned during the advancement of the plunger. During injection, the operator usually employs a one-handed technique. The operator steadies the barrel by holding two laterally-extending tabs at the trailing end of the barrel with the index and middle fingers and by using the thumb to advance the plunger in a distal-to-proximal (trailing-to-leading) direction in a well-known way.

There are occasions, however, when it is necessary to inject substances in a precise location, such as a patient's eye. For example, in order to inject into the appropriate area in or near the eye, an ophthalmologist must look closely into the eye using a slit lamp microscope. In this regard, the ophthalmologist uses one hand to stabilize and focus the microscope, while the other hand must simultaneously hold open the patient's eye and carefully inject the substance via the syringe needle.

Accordingly, when performing such an injection, the ophthalmologist must hold the syringe at the very tip near the needle, using the thumb and middle finger. The pinky and ring finger are used to hold open the eye, while the index finger stretches to the back of the syringe to push the plunger. In this manner, the index finger stretches to an unsafe distance in such a manner that control of the rate of injection is difficult by the ophthalmologist. In some instances, the ophthalmologist may not even be able to stretch the index finger a sufficient distance to reach the end of the plunger. As such, the ophthalmologist may move the hand stabilizing the microscope therefrom to aid in injection. However, the microscope may thereafter become unfocused if the patient moves, thereby limiting the ophthalmologist's visibility.

As such, it would be desirable to provide an apparatus and associated method capable of facilitating one-handed control during an injection procedure of any kind including the above, while also being capable of retrofitting to a conventional syringe so as to facilitate injection with one hand in the absence of any need to place the fingers, thumb or palm of the operator in an awkward position.

### BRIEF SUMMARY OF THE DISCLOSURE

The above and other needs are met by the present disclosure which, according to one aspect, provides a plunger control device adapted for attachment to a syringe including a syringe barrel having a barrel flange, and further including a syringe plunger having a leading end slideably disposed within the syringe barrel and a trailing end external to the syringe barrel, wherein the syringe plunger has a plunger flange disposed about the trailing end thereof. The plunger control device comprises an attachment member adapted to fixedly engage a syringe barrel. The plunger control device further comprises a translating member operably engaged with the attachment member. The translating member has an elongate guide member slideably engaged with the attachment member such that the translating member is slideable with respect thereto between a proximal end and a distal end of the elongate guide member. The elongate guide member is adapted to longitudinally extend along at least a portion of the syringe barrel when the attachment member is fixedly engaged thereto such that the elongate guide member is exterior to the syringe barrel. The translating member further has a plunger attachment portion disposed about the distal end of the elongate guide member and adapted to receive a portion of a syringe plunger for coupling thereto. The translating member further includes an engagement member extending from the proximal end of the elongate guide member so as to be spaced-apart from the syringe barrel when the attachment member is engaged with the syringe barrel.

Another aspect of the present disclosure provides a method for plunging a syringe including a syringe barrel having a barrel flange, wherein the syringe further includes a syringe plunger having a leading end slideably disposed within the syringe barrel and a trailing end external to the syringe barrel, and wherein the syringe plunger has a plunger flange disposed about the trailing end thereof. The method comprises coupling a portion of syringe plunger to a plunger attachment portion of a plunger control device. The plunger control device includes a translating member operably engaged with an attachment member configured to fixedly engage a syringe barrel. The translating member has an elongate guide member slideably engaged with the attachment member such that the translating member is slideable with respect thereto between a proximal end and a distal end of the elongate guide member. The elongate guide member is adapted to longitudinally extend along at least a portion of the syringe barrel when the attachment member is fixedly engaged thereto such that the elongate guide member is exterior to the syringe barrel. The plunger attachment portion is disposed about the distal end of the elongate guide member. The translating member further includes an engagement member extending from the proximal end of the elongate guide member. The method further comprises coupling the attachment member of the plunger control device to the syringe barrel such that the attachment member is fixed with respect to the syringe barrel, wherein the engagement member is spaced-apart from the syringe barrel. The method further comprises advancing the engagement member in a plunging direction away from the fixed attachment member so as to plunge the syringe.

Another aspect of the present disclosure provides a plunger control device adapted for coupling to a syringe having a syringe barrel with a barrel flange. The plunger control device comprises an attachment member adapted to fixedly engage a syringe barrel. The plunger control device further comprises a translating member operably engaged with the attachment member. The translating member has an elongate guide member defining a longitudinal axis and being slideably engaged with the attachment member such that the translating member is slideable with respect thereto between a proximal end and a distal end of the elongate guide member. The translating member further includes a plunger member fixedly engaged with the elongate guide member such that a longitudinal axis of the plunger member is substantially parallel with the longitudinal axis of the elongate guide member. The plunger member has a leading end capable of being slideably disposed within the syringe barrel and a trailing end external to the syringe barrel. The translating member further comprises an engagement member extending from the proximal end of the elongate guide member so as to be spaced-apart from the syringe barrel when the attachment member is engaged with the syringe barrel. The engagement member is disposed laterally proximal to the leading end of the plunger member such that the engagement member and the leading end maintain proximity during translation of the translating member.

Another aspect of the present disclosure provides a method for plunging a syringe having a syringe barrel with a barrel flange. The method comprises disposing a plunger member of a plunger control device within a syringe barrel. The plunger control device comprises a translating member operably engaged with an attachment member configured to fixedly engage a syringe barrel. The translating member has an elongate guide member defining a longitudinal axis and being slideably engaged with the attachment member such that the translating member is slideable with respect thereto between a proximal end and a distal end of the elongate guide member. The plunger member is fixedly engaged with the elongate guide member such that a longitudinal axis of the plunger member is substantially parallel with the longitudinal axis of the elongate guide member. The plunger member has a leading end slideably disposed within the syringe barrel and a trailing end external to the syringe barrel. The translating member further comprises an engagement member extending from the proximal end of the elongate guide member and adapted to be spaced-apart from the syringe barrel. The engagement member is disposed laterally proximal to the leading end of the plunger member such that the engagement member and the leading end maintain proximity during translation of the translating member. The method further comprises coupling the attachment member to the syringe barrel such that the attachment member is fixed with respect to the syringe barrel, wherein the engagement member is spaced-apart from the syringe barrel. The method further comprising advancing the engagement member in a plunging direction away from the fixed attachment member so as to plunge the syringe.

Another aspect of the present disclosure provides a syringe comprising a syringe barrel having a barrel flange. A plunger has a leading end slideably disposed within the syringe barrel and a trailing end external to the syringe barrel. An attachment member is configured to fixedly engage a syringe barrel. A translating member is operably engaged with the attachment member. The translating member has an elongate guide member defining a longitudinal axis and being slideably engaged with the attachment member such that the translating member is slideable with respect thereto between a proximal end and a distal end of the elongate guide member. The plunger is fixedly engaged with the elongate guide member such that a longitudinal axis of the plunger is substantially parallel with the longitudinal axis of the elongate guide member. The translating member further comprises an engagement member extending from the proximal end of the elongate guide member and being spaced-apart from the syringe barrel. The engagement member is disposed laterally proximal to the leading end of the plunger member such that the engagement member and the leading end maintain proximity during translation of the translating member.

Aspects of the present disclosure thus provide advantages as otherwise detailed herein.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIGS. 1 and 2 are perspective views of a plunger control device coupled to a syringe in a plunged position, according to one aspect of the present disclosure;

FIG. 3 is a perspective view of a plunger control device coupled to a syringe in a retracted position, according to one aspect of the present disclosure;

FIG. 4 is a perspective view of a translating member of a plunger control device, according to one aspect of the present disclosure;

FIGS. 5 and 6 are perspective views of an attachment member of a plunger control device, according to one aspect of the present disclosure;

FIG. 7 is a perspective view of a plunger control device, according to one aspect of the present disclosure;

FIG. 8 is a perspective view of a plunger control device, according to another aspect of the present disclosure;

FIGS. 9 and 10 are perspective views of a plunger control device coupled to a syringe in a partially plunged position, wherein the plunger control device includes a plunger member, according to one embodiment of the present invention;

FIG. 11 is a perspective view of a translating member of a plunger control device, wherein the translating member includes a plunger member, according to one aspect of the present disclosure;

FIG. 12 is a perspective view of a plunger control device coupled to a syringe, wherein a grip portion covers a portion of a syringe barrel of the syringe, according to one aspect of the present disclosure;

FIGS. 13 and 14 are perspective views illustrating the use of a plunger control device, according to one aspect of the present disclosure;

FIGS. 15 and 16 are perspective views of a plunger control device coupled to a syringe, the plunger control device having an engagement member capable of facilitating injection and retraction of the syringe, according to one aspect of the present disclosure; and

FIG. 17 is a perspective view of a plunger control device coupled to a syringe, the plunger control device having an engagement member capable of facilitating injection and retraction of the syringe, according to another aspect of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, this disclosure may be embodied in many different forms and should not be construed as limited to the aspects set forth herein; rather, these aspects are provided so that this disclosure will satisfy applicable legal requirements. Like numbers refer to like elements throughout.

Aspects of the present disclosure address concerns in which one-handed control of a plunger of a syringe is difficult due to physical limitations of the operator, as previously described. In this regard, aspects of the present disclosure improve ease of use for plunging the plunger of a syringe during an injection procedure. However, aspects of the present disclosure may also provide improved ease of use for retracting the syringe. In any event, aspects of the present disclosure provide apparatuses and methods for facilitating improved one-handed control of the plunger of a syringe.

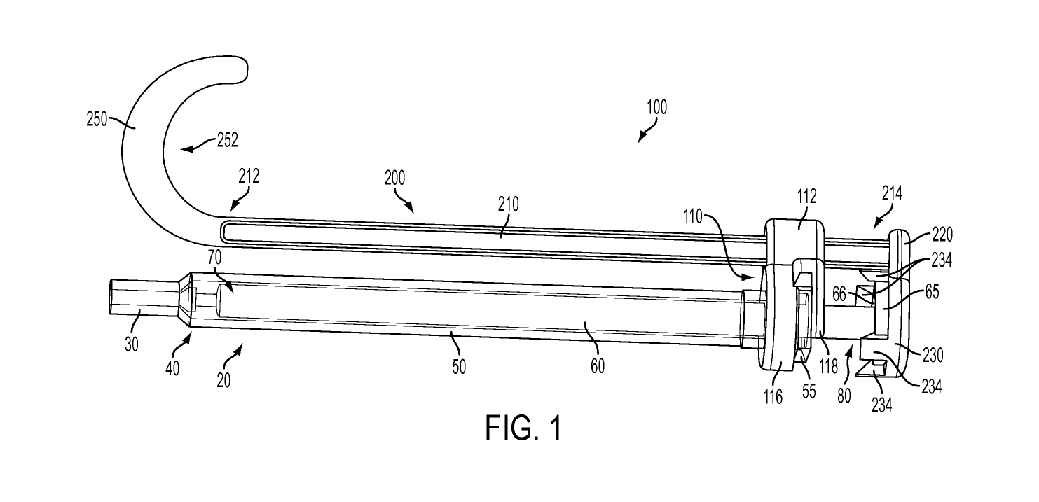

FIGS. 1-3, 7, 8 and 12 illustrate a plunger control device 100 for use with a syringe 20. The syringe 20 includes a needle (not shown) held at its base by a hub 30 that is mounted at the leading end 40 of a syringe barrel 50. According to some aspects, the hub 30 may include a luer-lock for securely receiving the needle. The hub 30 may be secured to the leading end 40 of the syringe barrel 50 and is configured to hold the base of the needle. In some instances, a barrel flange 55 may be provided wherein the barrel flange 55 extends laterally outward of the syringe barrel 50. A syringe plunger 60 is slideably mounted in the syringe barrel 50. A leading end 70 of the syringe plunger 60 is slideably disposed within the syringe barrel 50 and a trailing end 80 is external to the syringe barrel 50. An annular sealing member (not shown) is provided at the leading end 70 of the syringe plunger 60. The annular sealing member seals the syringe barrel 50 such that liquid drawn into the syringe barrel 50 during aspiration does not leak therepast, while also preventing liquid from leaking during injection. The syringe plunger 60 includes a plunger flange 65 for providing a finger/hand engagement section used to retract and advance the syringe plunger 60.

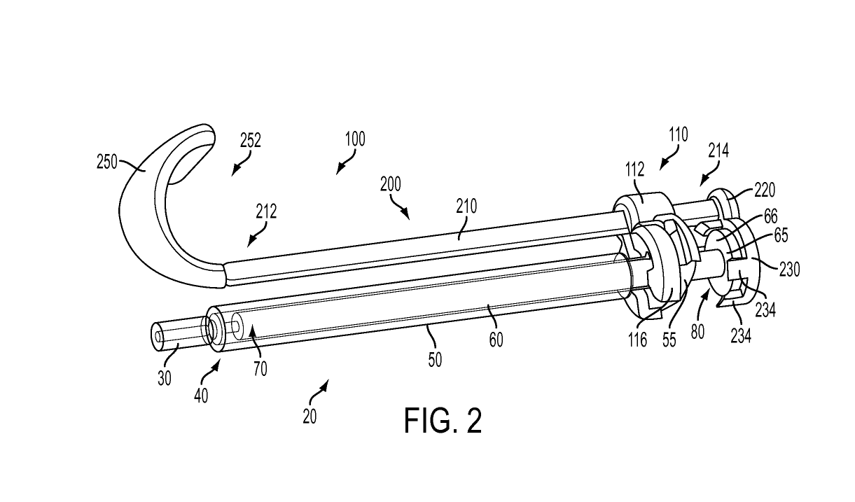

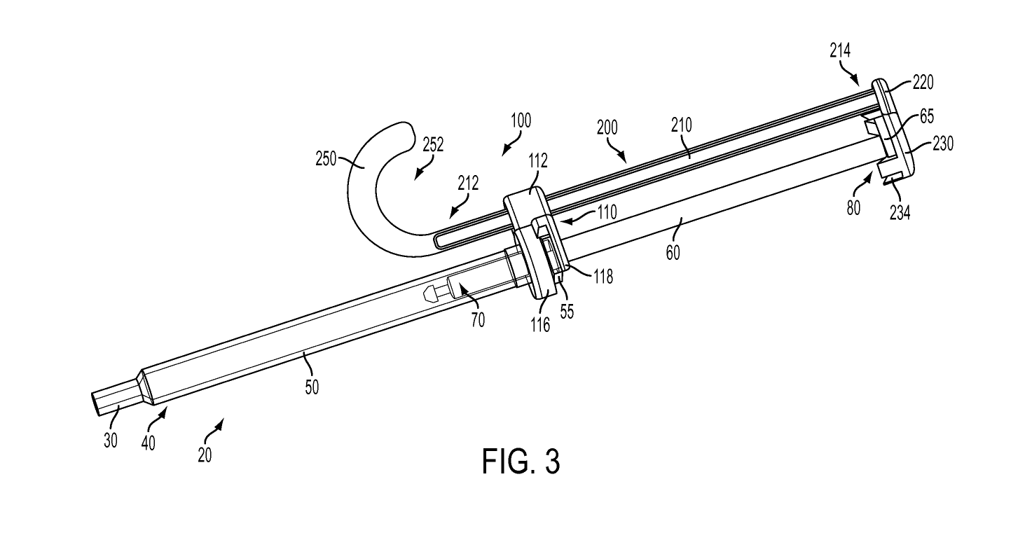

The plunger control device 100 is depicted with an advanced syringe plunger 60 in FIGS. 1 and 2 and with a retracted syringe plunger 60 in FIG. 3. In some instances, the plunger control device 100 may be retrofitted onto the syringe 20 so as to enable single-handed operation thereof. According to one aspect, the plunger control device 100 may include an attachment member 110 capable of fixedly engaging the syringe barrel 50. In some instances, the attachment member 110 may fasten to the syringe barrel 50 at the barrel flange 55. The attachment member 110 may be fixedly attached, fastened, or otherwise coupled to the syringe barrel 50 or the barrel flange 55, for example, using an interference or snap fit, such that the attachment member 110 does not slide or otherwise translate with respect to the syringe barrel 50. The attachment member 110 may be formed of a polymer or plastic material (e.g., a blend of polyphenylene oxide (PPO) and polystyrene (PS); available as NORYL from SABIC Innovative Plastics), or any other suitable material such as, for example, a ceramic or metal material. In some instances, the attachment member 100 may be provided as an integral portion of the syringe 20 or the syringe barrel 50. That is, the attachment member 100 may be integrally formed with the syringe barrel 50 such that the attachment member 100 is not provided as a separate and discrete component requiring attachment to the syringe 20 or syringe barrel 50.

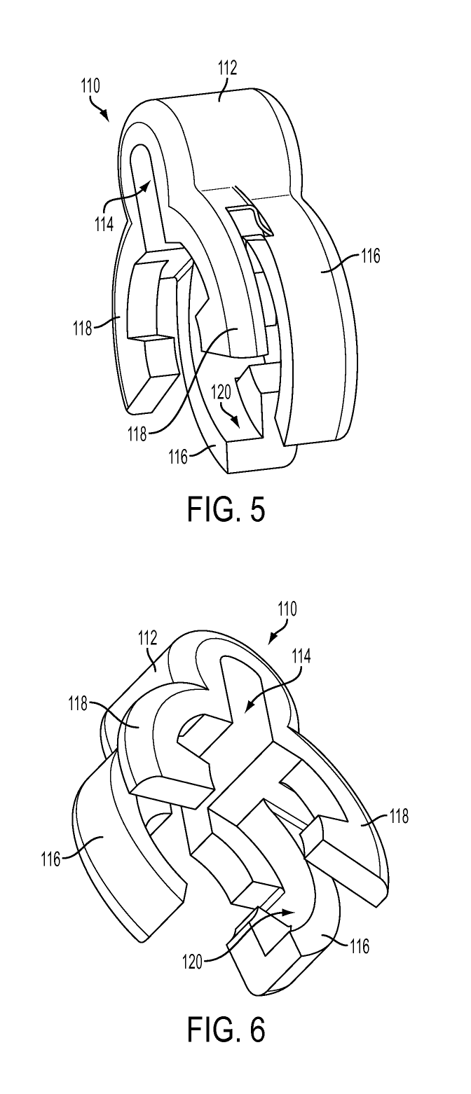

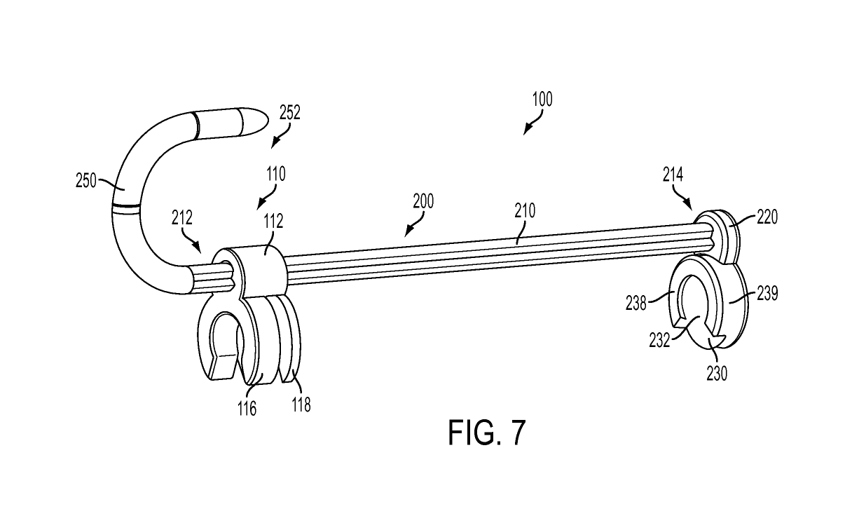

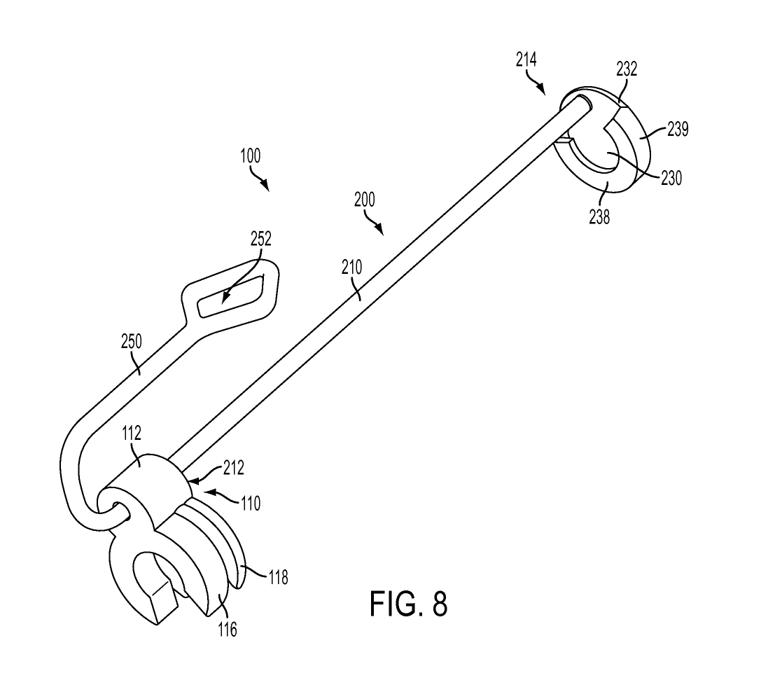

As shown in FIGS. 5 and 6, the attachment member 110 may include a first portion 112 defining a channel 114 extending therethough. In some instances, the first portion 112 may be substantially arcuately shaped, although aspects of the present disclosure are not limited to such a configuration. In some instances, as shown in FIGS. 2, 5, and 6, the channel 114 may be open-ended, while in other instances, as shown in FIGS. 7 and 8, the channel 114 may be closed (but still extending entirely through the first portion 112). The attachment member 110 may define channel 114 so as to have any desired configuration. For example, the attachment member 110 may define the channel 114 as substantially rectangular, rectilinear, or otherwise as having at least one pair of opposing parallel sidewalls such than an object extending through the channel 114 is not capable of rotating within the channel 114, as further described herein. According to another aspect, as shown in FIG. 7, the attachment member 110 may define overlapping cylindrical sections forming the channel 114 extending through the attachment member 110 to provide a track channel.

First and second arm members 116, 118 may extend from the first portion 112 in a spaced-apart arrangement such that the barrel flange 55 can be received therebetween. According to one aspect, the first and second arm members 116, 118 may have a substantially arcuate profile so as to accommodate the cylindrical shape of the syringe barrel 50 and the syringe plunger 60. In this regard, the first arm member 116 may be fixedly engaged with the syringe barrel 50. In some instances, the first arm member 116 may define a recessed portion 120 configured to accommodate a beveled portion (not shown) of the syringe barrel 50. That is, the syringe barrel 50 may have a beveled portion integrally connecting the barrel flange 55 to the remaining portion of the syringe barrel 50. As such, the recessed portion 120 may accommodate the increased diameter of the beveled portion such that a secure fit between the attachment member 110 and the syringe barrel 50 can be achieved.

The plunger control device 100 may further include a translating member 200. The translating member 200 may be slideably engaged with the attachment member 110. That is, the translating member 200 may be slideable with respect to the attachment member 110. In this regard, the attachment member 110 may be fixed to the syringe barrel 50 such that the attachment member 110 does not slide or otherwise translate with respect to the syringe barrel 50. The translating member 200 may be formed of a polymer or plastic material (e.g., a blend of polyphenylene oxide (PPO) and polystyrene (PS); available as NORYL from SABIC Innovative Plastics), or any other suitable material.

The translating member 200 may be fixedly coupled to, fastened to or otherwise engaged with the syringe plunger 60 such that the translating member 200 can control movement of the syringe plunger 60 with respect to the syringe barrel 50. In this manner, both the translating member 200 and the syringe plunger 60 translate with respect to the attachment member 110 and the syringe barrel 50. According to one aspect, the translating member 200 may attach, fasten or otherwise engage the syringe plunger 60 at or about the plunger flange 65. In other instances, the translating member 200 may fixedly engage the syringe plunger 60 at portions other than the plunger flange 65. In any event, the translating member 200 and the syringe plunger 60 are fixed with respect to one another, and the attachment member 110 and the syringe barrel 50 are fixed with respect to one another.

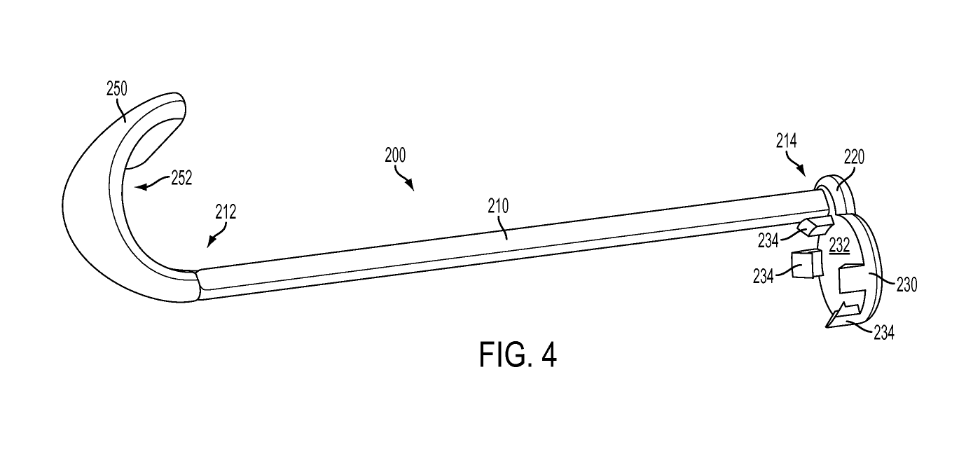

As shown in FIG. 4, the translating member 200 may include an elongate guide member 210 slideably engaged with the attachment member 110. In this regard, the elongate guide member 210 may move with respect to the attachment member 110 between a proximal end 212 and a distal end 214 of the elongate guide member 210. In some instances, the elongate guide member 210 may extend through the channel 114 of the attachment member 110 to provide the slideable arrangement. In this manner, the elongate guide member 210 essentially acts as a track. One of ordinary skill in the art will appreciate other mechanisms for slideably coupling the attachment member 110 to the elongate guide member 210. The elongate guide member 210 defines a longitudinal axis, which may be parallel with a longitudinal axis of the syringe barrel 50 when the attachment member 110 is engaged with the syringe barrel 50 such that the elongate guide member 210 extends longitudinally along the syringe barrel 50, and such that the elongate guide member 210 is exterior to the syringe barrel 50. In this instance, the elongate guide member 210 and the syringe barrel 50 are connected (via the attachment member 110) and arranged in parallel, wherein the elongate guide member 210 may be substantially equidistantly spaced-apart from the syringe barrel 50 along the entire length thereof.

In instances where the channel 114 is open-ended (e.g., FIGS. 2, 5 and 6), the elongate guide member 210 may be fit into the channel 114 in a direction perpendicular to a longitudinal axis defined by and common to the elongate guide member 210 and the channel 114. That is, the attachment member 110 may be slid over the elongate guide member 110 and attached thereto via an interference or snap fit. In this manner, the elongate guide member 210 and the attachment member 110 may be engaged after fabrication of the entire translating member 200. Further, the attachment member 110 may be removed from the translating member 200 at any time. In instances where the channel is closed (e.g., FIGS. 7 and 8), the attachment member 110 may need to be slid onto the elongate guide member 210 before fabrication of the translating member 200 is complete. In such instances, the attachment member 110 may not be easily removed from the translating member 200.

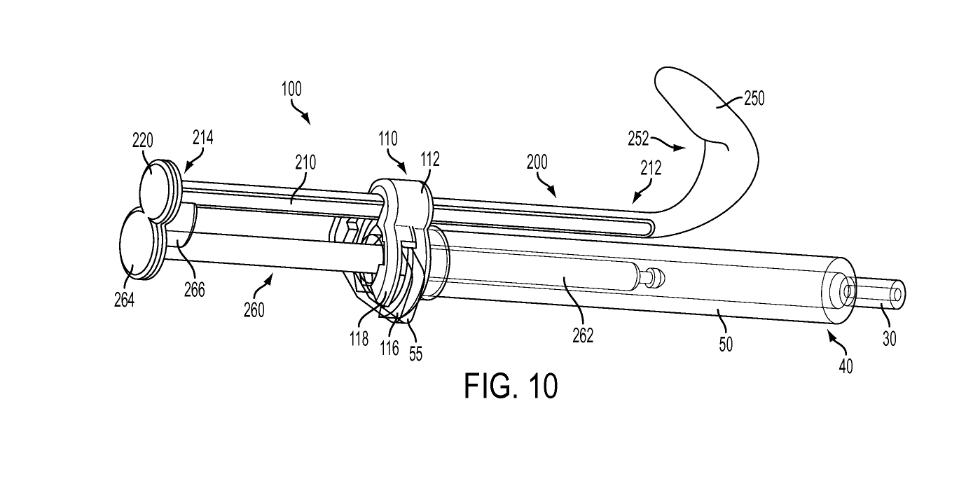

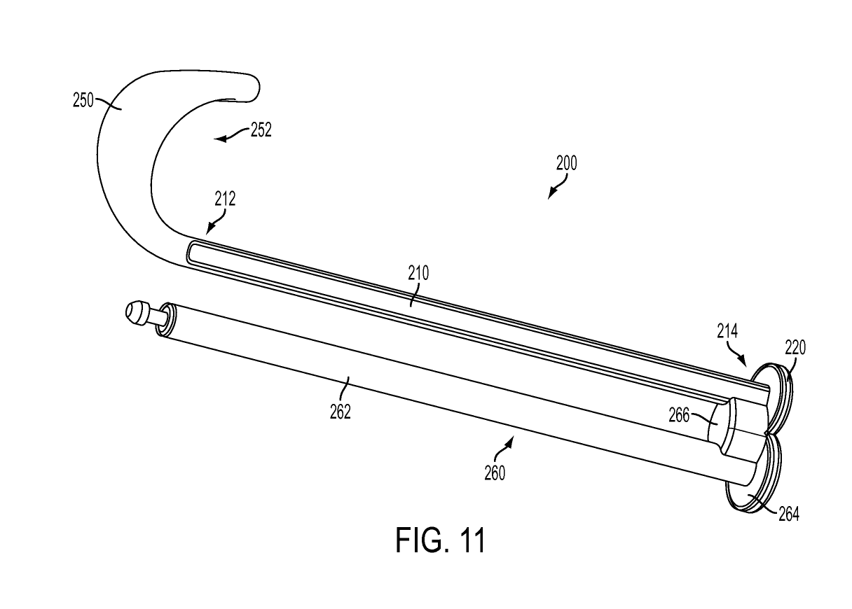

The attachment member 110 may define the channel 114 in any desired configuration such that the shape of the elongate guide member 210 can be accommodated and received therein. For example, the elongate guide member 210 may have a rectilinear profile (FIGS. 1-4 and 9-12), a barbell profile (FIG. 7), a cylindrical profile (FIG. 8), or any other suitable configuration. Furthermore, the elongate guide member 210 may be constructed of either a rigid or non-rigid material. According to some aspects, the channel 114 and the elongate guide member 210 correspond so as to cooperate to prevent the elongate guide member 210 from rotating with respect to the attachment member 110. For example, the rectilinear profile (FIGS. 1-4 and 9-12) and the barbell profile (FIG. 7) cooperate to substantially limit the rotation of the attachment member 110 with respect to the elongate guide member 210. The elongate guide member 210 may have any suitable configuration for limiting rotation with respect to the attachment member 110 in addition to those described herein.

The translating member 200 may include a stop or detent member 220 disposed at the distal end 214 of the elongate guide member 210 to prevent the attachment member 110 from disengaging the elongate guide member 210 at the distal end 214. In some instances, the detent member 220 may be integrally formed with the elongate guide member 210 as a single workpiece.

According to some aspects of the present disclosure, the translating member 200 may include a plunger attachment portion 230 in fixed engagement with the elongate guide member 210. The plunger attachment portion 230 may couple, fasten, or otherwise attach a portion of the syringe plunger 60 to the translating member 200. In one embodiment, the plunger attachment portion 230 may couple, fasten, or otherwise attach the plunger flange 65 of the syringe 20 to the translating member 200. For example, in one instance, the plunger attachment portion 230 may be fixedly engaged with the detent member 220, which is fixedly engaged with the elongate guide member 210, such that the plunger attachment portion 230 extends laterally outward of the elongate guide member 210 and substantially perpendicular to the longitudinal axis thereof. As such, translation of the elongate guide member 210 may effect translation of the plunger attachment portion 230 and, when attached, the syringe plunger 60.

According to one aspect, the plunger attachment portion 230 may be integrally formed with the detent member 220 as a single workpiece. However, in some instances, the plunger attachment portion 230 may be coupled to the detent member 220 with an appropriate fastener or fastening mechanism. Still, in some instances, the plunger attachment portion 230 may be directly engaged with the elongate guide member 210 (integrally formed or with appropriate fasteners) and may even provide the stop mechanism itself for preventing the attachment member 110 from disengaging the elongate guide member 210 at the distal end 214, as shown in FIG. 8.

The plunger attachment portion 230 may include a lateral surface 232 for abutting an opposing surface of the plunger flange 65 of the syringe 20. The interaction between these two surfaces creates the contact plane causing axial movement of the syringe plunger 60 (when plunged) or the translating member 200 (when retracted).

The plunger attachment portion 230 may have various arrangements for receiving the plunger flange 65 of the syringe 20 in a secured and fixed manner. For example, as shown in FIGS. 1-4 and 12, the plunger attachment portion 230 may have a plurality of tab portions 234 extending perpendicularly from the lateral surface 232 and about the periphery thereof. The tab portions 234 may be configured to provide an interference or snap fit with the plunger flange 65. Further, the tab portions 234 may be configured to clip around the plunger flange 65 wherein a portion of the tab 234 extends inwardly so as to engage a surface 66 of the plunger flange 65 opposite the surface of the plunger flange 65 proximate to the lateral surface 232 of the plunger attachment portion 230. In another exemplary aspect, as shown in FIGS. 7 and 8, the plunger attachment portion 230 may have define a pocket 236 for receiving the plunger flange 65 therein, wherein the plunger flange may be slid into the pocket 236. In this regard, the plunger attachment portion 230 may have a horseshoe or U-shaped portion 238 spaced-apart from and integral with the lateral surface 232 via a sidewall 239. The horseshoe portion 238 may be configured to accommodate the portion of syringe plunger 60 proximate to the plunger flange 65. One of ordinary skill in the art will appreciate other configurations for securely attaching the plunger flange 65 and/or the syringe plunger 65 to the plunger attachment portion 230.

The translating member 200 may include an engagement member 250 fixedly engaged with the elongate guide member 210. The engagement member 250 may be integrally formed with the elongate guide member 210, or the engagement member 250 may otherwise be attached, coupled, or fastened to the elongate guide member 210 in a suitable manner (i.e., using fasteners). In some instances, the engagement member 250 may extend from the proximal end 212 of the elongate guide member 210. In other instances, the engagement member 250 may extend laterally outward from a side(s) of the elongate guide member 210. When the plunger control device 100 is coupled to the syringe 20, the engagement member 250 is spaced-apart from the syringe barrel 50. Further, the engagement member 250 remains spaced-apart from the syringe barrel 50 during translation of the elongate guide member 210. By disposing the engagement member 250 at or proximate to the proximal end 212 of the elongate guide member 210, an operator can control the plunging action of the syringe plunger 60 in a manner that does not require the operator to advance the syringe plunger 60 by directly interacting with the plunger flange 65. According to some aspects, the engagement member 250 may be disposed proximate to the leading end 70 of the syringe plunger 60 so that during translation of the translating member 200 the engagement member 250 and the leading end 70 maintain proximity. Due to the spaced-apart nature of the engagement member 250 and the leading end 70, the two components maintain lateral proximity during translation of the translation member 200. For example, the engagement member 250 and the leading end 70 may maintain a lateral off-set of about 1 cm to about 5 cm during translation of the translation member 200.

The engagement member 250 provides an interaction point for the operator to use a finger or other portion of the hand to effect translation of the translating member 200. That is, the engagement member 250 may be configured for digital manipulation or engagement by the operator, wherein one or more of the operator's digits (i.e., fingers) can be used to engage the engagement member 250 for imparting translational movement thereto. In this regard, the engagement member 250 may be particularly configured to provide a contact point/surface that is structured for an operator's finger(s) or hand so as to facilitate a plunging or advancing action of the syringe plunger 60. According to one aspect, the engagement member 250 may have an arcuate or hook profile, wherein the arcuate profile defines a concave portion 252 extending toward the distal end 214 of the elongate guide member 210. Further, according to one exemplary aspect, the engagement member 250 may have an arcuate fantail configuration, wherein the engagement member 250 widens as it extends from the elongate guide member 210, as shown in FIGS. 1-4 and 9-12. In some instances, the engagement member 250 may be open-ended so as to facilitate access to the contact portion with which the operator will interact. In other instances, as shown in FIG. 8, the engagement member 250 may be configured or a portion thereof may be configured in a closed-loop configuration in which the operator must extend a finger(s) within the opening defined by the closed-loop.

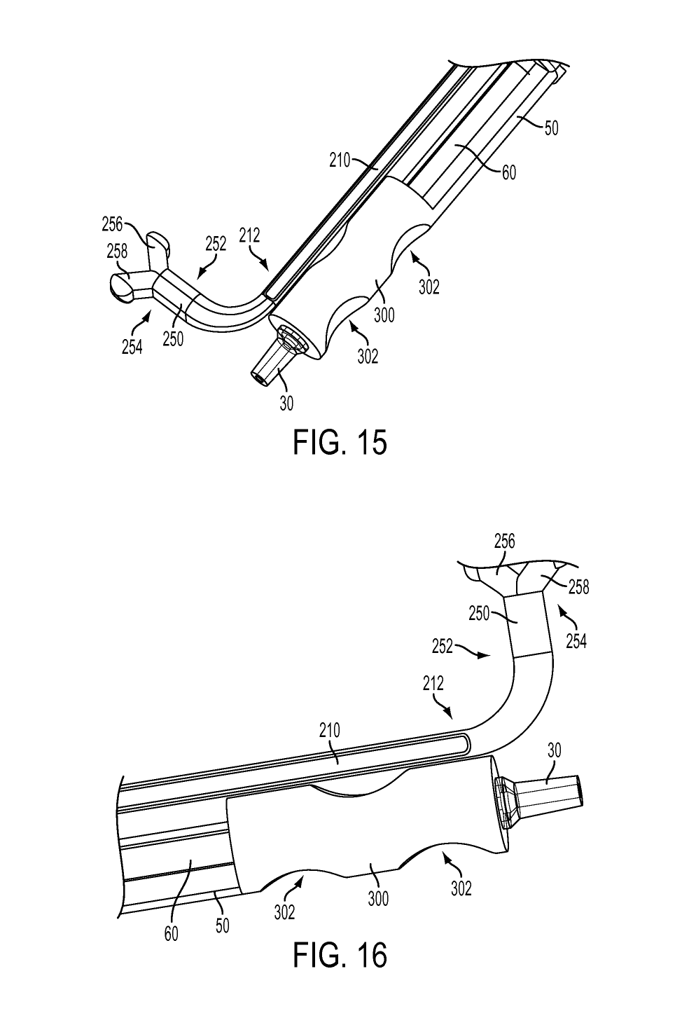

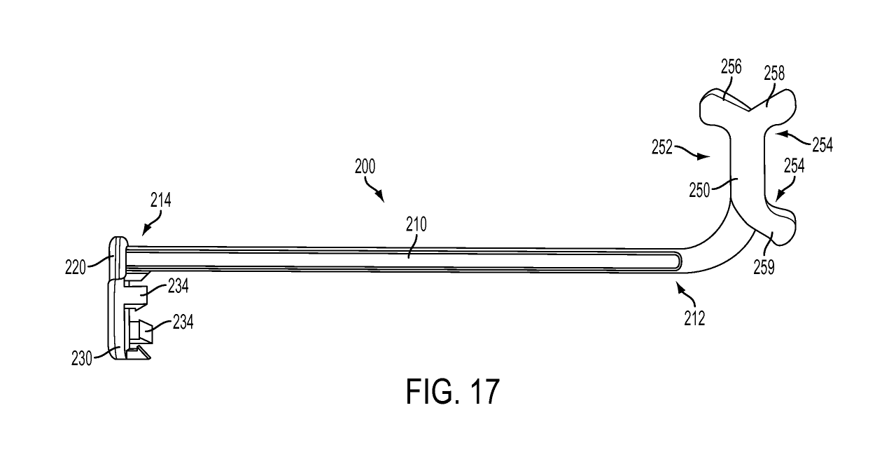

According to other aspects of the present disclosure, the engagement member 250 may be further configured to facilitate retraction of the syringe plunger 60, as shown in FIGS. 15, 16 and 17. In this regard, the engagement member 250 may include one or more supplemental arcuate portions 254 extending concavely away from the distal end 214 of the elongate guide member 210 for facilitating retraction of the syringe plunger 60. According to one exemplary aspect, the engagement member 250 may bifurcate at a terminal end thereof such that a first leg 256 cooperates with the remaining portions of the engagement member 250 to form the concave portion 252, while a second leg 258 cooperates with the remaining portions of the engagement member 250 to form the supplemental arcuate portion 254. In some instances, a third leg 259 (FIG. 17) may be provided to form an additional supplemental arcuate portion 254. In this manner, the translating member 200 may be capable of improving ease of use for both plunging and retraction of the syringe plunger 60. The configuration of the engagement member 250 to facilitate retraction may be provided in various arrangements, such as those provided to assist with the plunging action, as previously described.

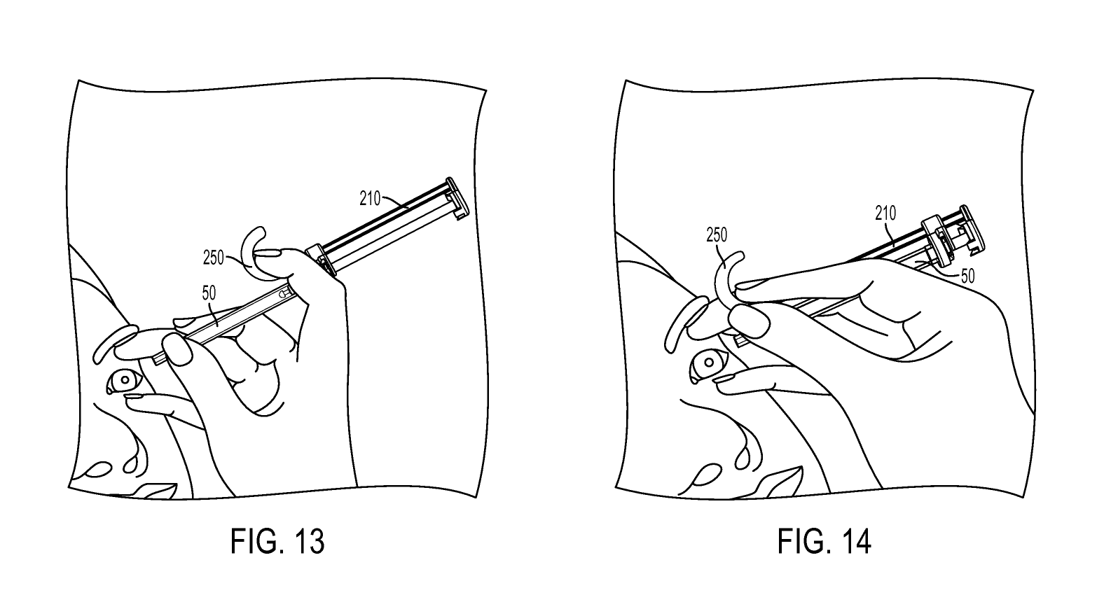

In operation, with reference to FIGS. 13 and 14, a syringe 20 may be provided. The plunger control device 100 may be coupled to the syringe 20 by snapping the plunger attachment portion 230 onto the plunger flange 65 of the syringe plunger 60. The attachment member 110 may be snapped onto the syringe barrel 50 by directing the attachment member 110 into engagement with the barrel flange 55 such that the barrel flange 55 is disposed between the first and second arm members 116, 118. At this point, a needle may be attached to the syringe 20, if necessary. The syringe plunger 60 may be retracted by the operator at either the plunger flange 65 or using the engagement member 250 so as to draw a liquid substance into the syringe barrel 50 for injection. In some instances, the needle may be removed and a new needle attached. The device and syringe assembly may then be positioned in front of a patient's eye.

The syringe barrel 50 may be held by the operator at the leading end 40 of a syringe barrel 50 using the middle finger and thumb. The device and syringe assembly may be held such that the engagement member 250 is on the side of the syringe 20 closest to the operator. The operator may hold the patient's eye open with the pinky and ring finger, and enter the tissue to be injected with the needle. The index finger of the operator may be positioned to engage the engagement member 250. The operator may then controllably advance the engagement member 250 toward the patient's eye, thereby advancing and plunging the syringe plunger 60 for injecting the liquid substance into the tissue. In some instances, the engagement member 250 may be advanced all the way over the thumb in order to fully inject the liquid substance. As such, the operator is able to provide improved single-handed control of the syringe, while keeping the other hand on the microscope for maintaining stabilization thereof for viewing the patient's eye. While the operation described herein is directed to an eye injection procedure, the plunger control device 100 of the present disclosure may be used with syringes to perform any number of procedures requiring plunging and/or retraction of a syringe. That is, the present disclosure may be used with syringes to perform any procedure requiring depression or withdrawal of a plunger, which includes, but is not limited to, any procedure requiring injection or aspiration of a material. For example, the present disclosure may be implemented in ophthalmic injections (e.g., retina (diabetic retinopathy), glaucoma, plastics injections (botox and cosmetic fillers)), insulin injections, hypodermic injections, blood withdrawals, dermatologic procedures (e.g., anesthesia), and veterinary applications.

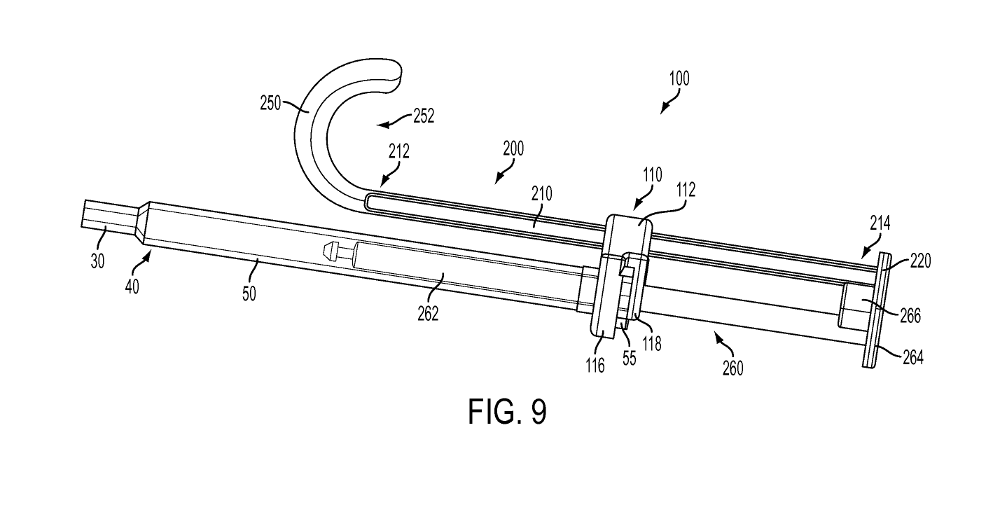

According to another aspect of the present disclosure, as shown in FIGS. 9-11, the plunger control device 100 may be capable of use with a syringe 20 not having a syringe plunger 60 (thus, also not having a plunger portion or plunger flange 65 for which to attach the plunger attachment portion 230). In this regard, the translating member 200 may include a plunger member 260 configured to be received within a syringe barrel 50 so as to serve the function of the syringe plunger of a conventional syringe. The plunger member 260 may have a cylindrical plunger portion 262 and a flange 264. The cylindrical plunger portion 262 may be integrally formed with the flange 264 or otherwise coupled thereto with an appropriate fastening mechanism. The plunger member 260 may be coupled, fastened, or otherwise attached to the elongate guide member 210 and/or the detent member 220 (when provided). For example, the flange 264 may be integrally formed with the detent member 220 so as to fixedly couple the elongate guide member 210 to the cylindrical plunger portion 262. Still, in some instances, the flange 264 may be directly engaged with the elongate guide member 210 (integrally formed or with appropriate fasteners) and may even provide the stop mechanism itself for preventing the attachment member 110 from disengaging the elongate guide member 210 at the distal end 214. In some instances, a connector member 266 may be provided to provide structural integrity to the section in which the elongate guide member 210 and the plunger member 260 are connected.

Operation of the plunger control device 100 employing the plunger member 260 may be similarly effectuated as with the plunger control device 100 configured to engage the syringe plunger 60 of the syringe 20, as previously described herein. In this manner, according to some aspects, the engagement member 250 may be disposed proximate to the leading end of the plunger portion 262 so that during translation of the translating member 200 the engagement member 250 and the leading end maintain proximity. Due to the spaced-apart nature of the engagement member 250 and the leading end, the two components maintain lateral proximity during translation of the translation member 200. For example, the engagement member 250 and the leading end may maintain a lateral off-set of about 1 cm to about 5 cm during translation of the translation member 200.

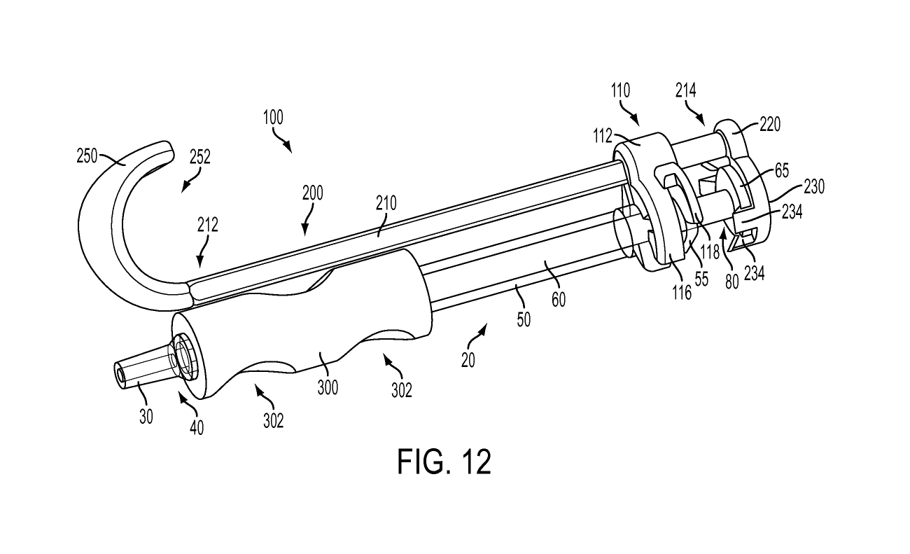

According to another aspect of the present disclosure, as shown in FIGS. 12, 15 and 16, a grip member 300 may be disposed about the syringe barrel 50 so as to extend at least partially along a length thereof. The grip member 300 may provide a gripping surface for improved grip of the device and syringe assembly during an injection or retraction procedure. In some instances, the grip member 300 may provide indicia of gripping positions to an operator. For example, the grip member 300 may define a plurality of concave portions 302 that provide indicia of gripping positions to the operator. That is, the concave portions 302 may indicate that a user should position a finger within the concave portion 302.

Many modifications and other aspects of the disclosure set forth herein will come to mind to one skilled in the art to which this disclosure pertains having the benefit of the teachings presented in the foregoing descriptions and the associated drawings. For example, in some instances, the translating member 200 and/or the elongate guide member 210 may be configured to provide increased leverage ratios through the use of for example, pulleys, levers, gears, etc. In other instances, a ratcheting system may be implemented such that the translating member 200 is incrementally translated based on a ratcheting mechanism. Further, aspects of the present disclosure may include electronic displacement systems using, for example, a go-no-go mechanism or a force sensitive mechanism. Additionally, aspects of the present disclosure may be implemented into or otherwise comprise a portion of a larger structure, such as, for example, a slit lamp microscope/machine. Therefore, it is to be understood that the disclosure is not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. A plunger control device adapted for attachment to a syringe including a syringe barrel having a barrel flange, and the syringe further including a syringe plunger having a leading end slideably disposed within the syringe barrel and a trailing end external to the syringe barrel, the syringe plunger having a plunger flange disposed about the trailing end thereof, the plunger control device comprising:
an attachment member adapted to fixedly engage a syringe barrel; and
a translating member operably engaged with the attachment member, the translating member having an elongate guide member slideably engaged with the attachment member such that the translating member is slideable with respect thereto between a proximal end and a distal end of the elongate guide member, the elongate guide member being adapted to longitudinally extend along at least a portion of the syringe barrel when the attachment member is fixedly engaged thereto such that the elongate guide member is exterior to the syringe barrel, the translating member further having a plunger attachment portion disposed about the distal end of the elongate guide member and configured to operably engage a portion of a syringe plunger for coupling thereto, the translating member further comprising an engagement member extending from the proximal end of the elongate guide member so as to be spaced-apart from the syringe barrel when the attachment member is engaged with the syringe barrel.

2. The plunger control device according to claim 1, wherein the engagement member comprises an arcuate portion extending concavely toward the distal end of the elongate guide member for facilitating translation of the translating member.

3. The plunger control device according to claim 2, wherein the engagement member is substantially open-ended.

4. The plunger control device according to claim 2, wherein the engagement member comprises a supplemental arcuate portion extending concavely away from the distal end of the elongate guide member for facilitating retraction of the syringe plunger.

5. The plunger control device according to claim 1, wherein the attachment member defines a channel extending therethrough, the elongate guide member extending through the channel of the attachment member such that the translating member is slideable with respect thereto, and further wherein the elongate guide member defines a longitudinal axis substantially parallel with a longitudinal axis defined by the syringe barrel when the attachment member is engaged therewith such that the elongate guide member is adapted to be spaced-apart from the syringe barrel.

6. The plunger control device according to claim 5, wherein the channel defined by the attachment member is open-ended.

7. The plunger control device according to claim 5, wherein the attachment member comprises a first portion defining the channel and first and second arm members extending from the first portion so as to be substantially perpendicular with respect to the elongate guide member, the first and second arm members being spaced-apart so as to receive a barrel flange of the syringe barrel therebetween.

8. The plunger control device according to claim 7, wherein the first arm member is disposed between the plunger attachment portion and the second arm member, the second arm member defining a recessed portion configured to facilitate coupling of the barrel flange to the attachment member.

9. The plunger control device according to claim 5, wherein the elongate guide member comprises a track guide member having a shape corresponding to the shape of the channel of the attachment member, the track guide member being configured such that the attachment member is non-rotatable with respect thereto.

10. The plunger control device according to claim 9, wherein the track guide member has a substantially rectilinear profile.

11. The plunger control device according to claim 1, wherein the plunger attachment portion is configured to operably engage a plunger flange of the syringe plunger, the plunger attachment portion comprising a lateral surface adapted to abut the plunger flange, the plunger attachment portion further comprising a plurality of tab portions extending perpendicularly from the lateral surface, the tab portions being arranged to facilitate coupling of the plunger attachment portion and the plunger flange via an interference fit.

12. The plunger control device according to claim 1, wherein the plunger attachment portion is configured to operably engage a plunger flange of the syringe plunger, the plunger attachment portion comprising a lateral surface adapted to abut the plunger flange, the plunger attachment portion further defining an open-ended slot adapted to receive the plunger flange such that the plunger flange is capable of being coupled to the plunger attachment portion.

13. A method for plunging a syringe including a syringe barrel having a barrel flange, and the syringe further including a syringe plunger having a leading end slideably disposed within the syringe barrel and a trailing end external to the syringe barrel, the syringe plunger having a plunger flange disposed about the trailing end thereof, the method comprising:
coupling a portion of a syringe to a plunger attachment portion of a plunger control device, the plunger control device comprising a translating member operably engaged with an attachment member configured to fixedly engage a syringe barrel, the translating member having an elongate guide member slideably engaged with the attachment member such that the translating member is slideable with respect thereto between a proximal end and a distal end of the elongate guide member, the elongate guide member longitudinally extending along at least a portion of the syringe barrel such that the elongate guide member is exterior to the syringe barrel, the plunger attachment portion being disposed about the distal end of the elongate guide member, the translating member further comprising an engagement member extending from the proximal end of the elongate guide member;
coupling the attachment member of the plunger control device to the syringe barrel such that the attachment member is fixed with respect to the syringe barrel, the engagement member being spaced-apart from the syringe barrel; and
advancing the engagement member in a plunging direction away from the fixed attachment member so as to plunge the syringe.

14. The method according to claim 13, wherein advancing the engagement member in a plunging direction further comprises advancing the engagement member having an arcuate portion extending concavely toward the distal end of the elongate guide member for facilitating translation of the translating member.

15. The method according to claim 14, wherein the engagement member is substantially open-ended.

16. The method according to claim 13, further comprising retracting the syringe plunger by moving the plunger flange axially away from the syringe barrel prior to the step of advancing the engagement member so as to prepare the syringe for injection.

17. The method according to claim 13, wherein the attachment member defines a channel extending therethrough, the elongate guide member extending through the channel of the attachment member such that the translating member is slideable with respect thereto, and further wherein the elongate guide member defines a longitudinal axis substantially parallel with a longitudinal axis defined by the syringe barrel when the attachment member is engaged therewith such that the elongate guide member is adapted to be spaced-apart from the syringe barrel.

18. The method according to claim 17, wherein the elongate guide member comprises a track guide member having a shape corresponding to the shape of the channel of the attachment member, the track guide member being configured such that the attachment member is non-rotatable with respect thereto.

19. The method according to claim 17, further comprising coupling the attachment member to the elongate guide member by sliding the attachment member onto the elongate guide member via the channel.

20. The method according to claim 17, wherein the channel defined by the attachment member is open-ended, and wherein the method further comprises coupling the attachment member to the elongate guide member by fitting the attachment member over the elongate guide member via the open-ended channel.

21. The method according to claim 17, wherein coupling the attachment member to the syringe barrel further comprises receiving the barrel flange of the syringe barrel between first and second arm members of the attachment member, the first and second arm members extending from a first portion of the attachment member defining the channel, the first and second arm members being substantially perpendicular with respect to the elongate guide member.

22. The method according to claim 21, wherein the first arm member is disposed between the plunger attachment portion and the second arm member, the second arm member defining a recessed portion configured to facilitate coupling of the barrel flange to the attachment member.

23. The method according to claim 13, wherein the plunger attachment portion comprises a lateral surface configured to abut a plunger flange of the syringe plunger, the plunger attachment portion further comprising a plurality of tab portions extending perpendicularly from the lateral surface, the tab portions being arranged to facilitate coupling of the plunger attachment portion and the plunger flange via an interference fit.

24. The method according to claim 13, wherein the plunger attachment portion comprises a lateral surface adapted to abut a plunger flange of the syringe plunger, the plunger attachment portion further defining an open-ended slot adapted to receive the plunger flange such that the plunger flange is capable of being coupled to the plunger attachment portion.

25. A plunger control device adapted for coupling to a syringe having a syringe barrel with a barrel flange, the plunger control device comprising:
an attachment member adapted to fixedly engage a syringe barrel; and
a translating member operably engaged with the attachment member, the translating member having an elongate guide member slideably engaged with the attachment member such that the translating member is slideable with respect thereto between a proximal end and a distal end of the elongate guide member, the translating member further having a plunger member fixedly engaged with the elongate guide member such that a longitudinal axis of the plunger member is substantially parallel with the longitudinal axis of the elongate guide member, the plunger member having a leading end capable of being slideably disposed within the syringe barrel and a trailing end external to the syringe barrel, the translating member further comprising an engagement member extending from the proximal end of the elongate guide member so as to be spaced-apart from the syringe barrel when the attachment member is engaged with the syringe barrel, the engagement member being disposed laterally proximal to the leading end of the plunger member such that the engagement member and the leading end maintain proximity during translation of the translating member.

26. The plunger control device according to claim 25, wherein the engagement member comprises an arcuate portion extending concavely toward the distal end of the elongate guide member for facilitating translation of the translating member.

27. The plunger control device according to claim 26, wherein the engagement member is substantially open-ended.

28. The plunger control device according to claim 26, wherein the engagement member comprises a supplemental arcuate portion extending concavely away from the distal end of the elongate guide member for facilitating retraction of the syringe plunger

29. The plunger control device according to claim 25, wherein the attachment member defines a channel extending therethrough, the elongate guide member extending through the channel of the attachment member such that the translating member is slideable with respect thereto.

30. The plunger control device according to claim 29, wherein the channel defined by the attachment member is open-ended.

31. The plunger control device according to claim 29, wherein the elongate guide member comprises a track guide member having a shape corresponding to the shape of the channel of the attachment member, the track guide member being configured such that the attachment member is non-rotatable with respect thereto.

32. The plunger control device according to claim 29, wherein the attachment member comprises a first portion defining the channel and first and second arm members extending from the first portion so as to be substantially perpendicular with respect to the elongate guide member, the first and second arm members being spaced-apart so as to receive a barrel flange of the syringe barrel therebetween.

33. The plunger control device according to claim 32, wherein the first arm member is disposed between the plunger attachment portion and the second arm member, the second arm member defining a recessed portion configured to facilitate coupling of the barrel flange to the attachment member.

34. A method for plunging a syringe having a syringe barrel with a barrel flange, the method comprising:
disposing a plunger member of a plunger control device within a syringe barrel, the plunger control device comprising a translating member operably engaged with an attachment member configured to fixedly engage a syringe barrel, the translating member having an elongate guide member defining a longitudinal axis and being slideably engaged with the attachment member such that the translating member is slideable with respect thereto between a proximal end and a distal end of the elongate guide member, the plunger member being fixedly engaged with the elongate guide member such that a longitudinal axis of the plunger member is substantially parallel with the longitudinal axis of the elongate guide member, the plunger member having a leading end slideably disposed within the syringe barrel and a trailing end external to the syringe barrel, the translating member further comprising an engagement member extending from the proximal end of the elongate guide member and adapted to be spaced-apart from the syringe barrel, the engagement member being disposed laterally proximal to the leading end of the plunger member such that the engagement member and the leading end maintain proximity during translation of the translating member;
coupling the attachment member to the syringe barrel such that the attachment member is fixed with respect to the syringe barrel, the engagement member being spaced-apart from the syringe barrel; and
advancing the engagement member in a plunging direction away from the fixed attachment member so as to plunge the syringe.

35. The method according to claim 34, wherein advancing the engagement member in a plunging direction further comprises advancing the engagement member having an arcuate portion extending concavely toward the distal end of the elongate guide member for facilitating translation of the translating member.

36. The method according to claim 35, wherein the engagement member is substantially open-ended.

37. The method according to claim 34, further comprising retracting the plunger member by moving the trailing end of the plunger member axially away from the syringe barrel prior to the step of advancing the engagement member, so as to prepare the syringe for injection.

38. The method according to claim 34, wherein the attachment member defines a channel extending therethrough, the elongate guide member extending through the channel of the attachment member such that the translating member is slideable with respect thereto, and further wherein the elongate guide member defines a longitudinal axis substantially parallel with a longitudinal axis defined by the syringe barrel, the elongate guide member being spaced-apart from the syringe barrel.

39. A syringe comprising:
a syringe barrel having a barrel flange;
a plunger having a leading end slideably disposed within the syringe barrel and a trailing end external to the syringe barrel;
an attachment member configured to fixedly engage a syringe barrel; and
a translating member operably engaged with the attachment member, the translating member having an elongate guide member defining a longitudinal axis and being slideably engaged with the attachment member such that the translatable member is slideable with respect thereto between a proximal end and a distal end of the elongate guide member, the plunger being fixedly engaged with the elongate guide member such that a longitudinal axis of the plunger is substantially parallel with the longitudinal axis of the elongate guide member, the translating member further comprising an engagement member extending from the proximal end of the elongate guide member and being spaced-apart from the syringe barrel, the engagement member being disposed laterally proximal to the leading end of the plunger member such that the engagement member and the leading end maintain proximity during translation of the translating member.

40. The syringe according to claim 39, wherein the engagement member comprises an arcuate portion extending concavely toward the distal end of the elongate guide member for facilitating translation of the translating member.

41. The syringe according to claim 39, wherein the plunger is discrete from the translating member.

42. The syringe according to claim 39, further comprising a grip member disposed about the syringe barrel and extending at least partially along a length thereof.

43. The syringe according to claim 42, wherein the grip member is configured to provide indicia of gripping positions to an operator.

44. The syringe according to claim 39, wherein the attachment member is integrally-formed with the syringe barrel.
